(19) United States
(12) Patent Application Publication
(10) Pub. No.: US 2026/0253690 A1
(43) Pub. Date: Aug. 27, 2026

(54) SYSTEM

(71) Applicant: SoftBank Group Corp., Tokyo (JP)

(72) Inventor: Hiroki TANI, Tokyo (JP)

(73) Assignee: SoftBank Group Corp., Tokyo (JP)

(21) Appl. No.: 19/536,247

(22) Filed: Feb. 11, 2026

(30) Foreign Application Priority Data
Feb. 21, 2025 (JP) 2025-027069

(51) Int. Cl. G16H 10/60; G06F 21/62; G06V 40/16; G10L 15/183; G10L 15/30; G10L 25/30; G10L 25/63

(52) U.S. Cl. CPC G16H 10/60; G06F 21/6254; G10L 15/183; G10L 15/30; G10L 25/30; G06V 40/174; G10L 25/63

(57) ABSTRACT

A system according to the embodiment includes an analysis unit, a creation unit, and a conversion unit. The analysis unit analyzes an utterance of a patient. The creation unit automatically creates a medical record based on information obtained by the analysis unit. The conversion unit converts an explanation of a doctor into a tone of a character based on the medical record created by the creation unit.

### CROSS-REFERENCE TO RELATED APPLICATION

The present application claims priority to and incorporates by reference the entire contents of Japanese Patent Application No. 2025-027069 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there are cases where it is difficult for a patient to convey his/her symptoms to a doctor, or where an explanation from a doctor is difficult to be conveyed to the patient, and there is room for improvement.

### SUMMARY OF THE INVENTION

A system according to the embodiment includes an analysis unit, a creation unit, and a conversion unit. The analysis unit analyzes an utterance of a patient. The creation unit automatically creates a medical record based on information obtained by the analysis unit. The conversion unit converts an explanation of a doctor into a tone of a character based on the medical record created by the creation unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

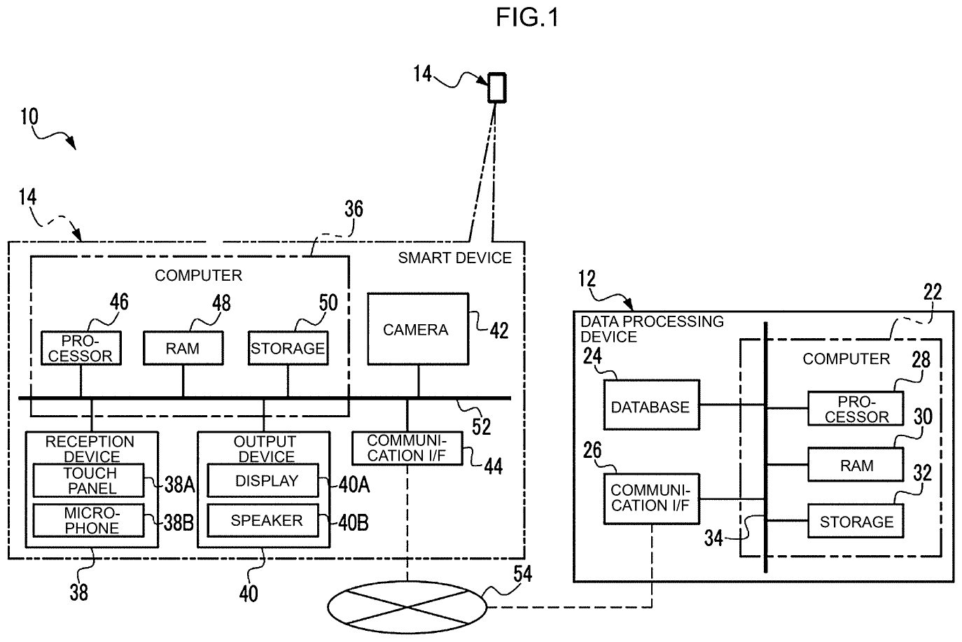

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

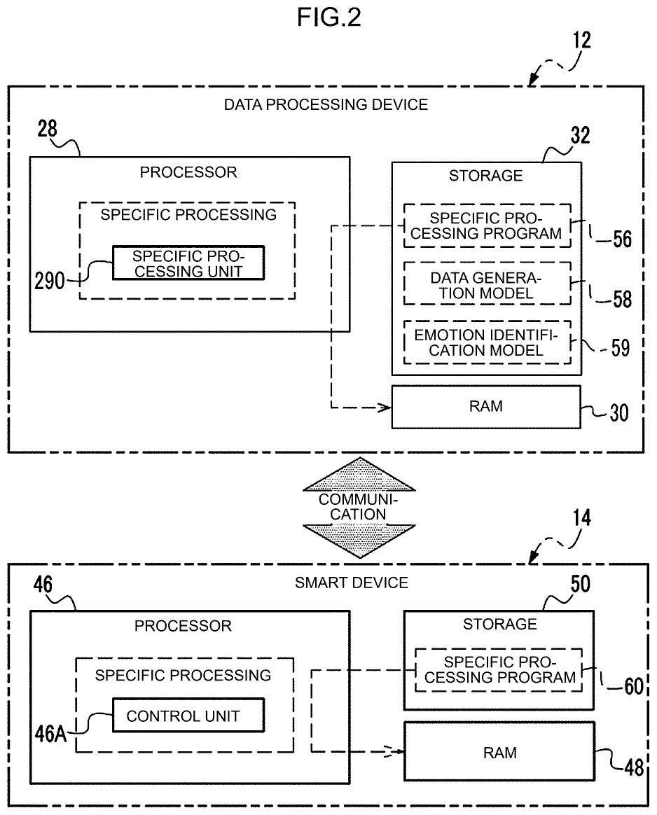

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The medical support system according to the embodiment of the present invention is a system configured to analyze an utterance of a patient, automatically create a medical record, and convert an explanation of a doctor into an easy-to-understand manner. In this medical support system, when a patient describes his/her symptom with a clumsy expression, a generative API analyzes the utterance of the patient and accurately grasps the symptom. For example, when the patient says “I have a stomachache,” the generative API analyzes the utterance and extracts detailed information such as a site, a degree, and an onset time of the pain. Next, the medical record is automatically created based on the information analyzed by the generative API. The symptom and the content of the utterance of the patient are described in detail in the medical record. Thereby, the doctor can accurately grasp the symptom of the patient. Furthermore, when the doctor gives an explanation to the patient, the generative API converts the explanation of the doctor into a tone of a character and conveys it to the patient in an easy-to-understand manner. For example, when the doctor says “Please take this medicine three times a day,” the generative API converts the explanation into the tone of the character and conveys it to the patient in a form such as “Please take this medicine three times a day, okay?” With this system, the patient can accurately convey his/her symptom to the doctor, and can easily understand the explanation from the doctor. In particular, it is a great help for patients who have difficulty communicating in a medical setting, such as children and the elderly. Thereby, the medical support system can analyze the utterance of the patient, automatically create the medical record, and convert the explanation of the doctor in an easy-to-understand manner. Specifically, the present medical support system is realized by an information processing apparatus integrating a sophisticated natural language processing algorithm and audio signal processing technology. The present system receives utterance voice data of the patient as an input, performs noise reduction and feature extraction on the data by digital signal processing, and then inputs the data to a multilayer neural network. The present system executes a slot filling process of extracting medical domain-specific entities such as “symptom,” “site,” “degree,” and “period” from input unstructured text data using a generative artificial intelligence model such as a large language model. The present system generates structured data compliant with a medical information exchange standard (HL7 FHIR, etc.) based on the extracted entities, and stores the data in a database of an electronic medical record system. The present system applies a Style Transfer Model to content of an utterance of the doctor to replace technical terms with plain expressions, and at the same time, generates a text reflecting a persona (vocabulary, sentence ending, tone) of a specified character. The present system inputs the generated text to a speech synthesis engine and outputs synthesized speech with adjusted emotion parameters to the patient. Thereby, the present system achieves a technical effect of not only recording and transmitting information but also resolving information asymmetry in medical communication and improving a level of understanding and a sense of security of the patient.

The medical support system according to the embodiment comprises an analysis unit, a creation unit, and a conversion unit. The analysis unit analyzes an utterance of a patient. The utterance of the patient includes, for example, voice data, text data, and the like, but is not limited to such examples. The analysis unit converts the utterance of the patient into text data using, for example, speech recognition technology, and analyzes content thereof. In addition, the analysis unit can analyze the utterance of the patient using a generative API to accurately grasp a symptom. For example, the generative API receives the utterance of the patient as an input and outputs detailed information of the symptom. The creation unit automatically creates a medical record based on information obtained by the analysis unit. In the medical record, for example, the symptom of the patient, content of the utterance, a medical history, and the like are described, but the medical record is not limited to such examples. The creation unit can also automatically create the medical record based on the information obtained by the analysis unit using a generative API. For example, the generative API receives the information obtained from the analysis unit as an input and outputs content of the medical record. The conversion unit converts an explanation of a doctor into a tone of a character based on the medical record created by the creation unit. The conversion unit can convert the explanation of the doctor into the tone of the character using, for example, a generative API, and convey it to the patient in an easy-to-understand manner. For example, the generative API receives the explanation of the doctor as an input and outputs the explanation converted into the tone of the character. Thereby, the medical support system according to the embodiment can analyze the utterance of the patient, automatically create the medical record, and convert the explanation of the doctor in an easy-to-understand manner. Specifically, the present analysis unit comprises a preprocessing module configured to receive voice waveform data as an input and extract acoustic features such as Mel-frequency cepstral coefficients (MFCC), and an automatic speech recognition (ASR) model configured to convert the extracted features into a text sequence. The present analysis unit applies a natural language understanding (NLU) model based on a Transformer architecture to the converted text data to perform context-dependent intent interpretation and key phrase extraction. The present creation unit receives structured data (for example, a symptom list in JSON format) output by the analysis unit as an input, and generates a natural sentence compliant with a SOAP format (Subjective data, Objective data, Assessment, Plan) using a pre-trained language generation model. The present conversion unit receives an utterance text of the doctor and an attribute vector (embedding representation) of a target character as inputs, and executes text style transfer using a Generative Adversarial Network (GAN) or a Diffusion Model. The present conversion unit outputs the converted text and sends a prosody control signal to a subsequent speech synthesis module. Thereby, in the present system, each processing unit cooperates highly by a deep learning model, realizing accurate and friendly exchange of medical information while reducing human cognitive load.

The medical support system comprises a selection unit configured to select an expression corresponding to an age or a level of understanding of the patient. The selection unit selects an appropriate expression corresponding to the age or the level of understanding of the patient. For example, the selection unit can select a friendly expression or a technical expression according to an age group of the patient. In addition, the selection unit can select a simple expression or a detailed explanation according to the level of understanding of the patient. For example, the selection unit can select a simple expression for children and select a detailed explanation for the elderly. Thereby, the medical support system can select an appropriate expression corresponding to the age or the level of understanding of the patient. Specifically, the present selection unit receives a user profile obtained by vectorizing attribute information of the patient (age, gender, past medical history, etc.) as an input. The present selection unit estimates a medical literacy level of the patient using a classification model, and calculates an estimation score thereof (for example, a continuous value ranging from 0 to 1). The present selection unit dynamically adds a constraint condition corresponding to the calculated literacy level (such as “use vocabulary of elementary school student level” or “add annotation to technical terms”) to an input prompt to a large language model. The present selection unit applies a scoring function for evaluating a degree of conformity to a target reader group to a plurality of generated expression candidates, and selects and outputs an expression having the highest score. The present selection unit uses reinforcement learning to feed back a reaction of the patient (an answer for checking the level of understanding or a change in facial expression) as a reward, and continuously updates a policy for expression selection. Thereby, the present system realizes dynamic and adaptive information presentation optimized for characteristics of an individual patient, rather than static rule-based conversion.

The medical support system comprises a protection unit configured to ensure accuracy of medical data and protect privacy. The protection unit ensures the accuracy of the medical data and protects the privacy. For example, the protection unit confirms accuracy and completeness of data in order to evaluate the accuracy of the data. In addition, the protection unit can perform encryption of data and access control for privacy protection. For example, the protection unit protects the privacy of the data by encrypting the data and setting an access authority. Thereby, the medical support system can ensure the accuracy of the medical data and protect the privacy. Specifically, the present protection unit applies a personal information protection filter to input medical data, automatically detects personally identifiable information (PII) such as a name, an address, and a telephone number, and performs masking or tokenization processing. The present protection unit uses a sophisticated encryption algorithm such as AES-256 in storage and transmission of data to ensure confidentiality of the data. The present protection unit generates a hash value of data using a hash function (SHA-256, etc.) to verify the completeness of the data, and collates it with a hash value recorded in a blockchain or a tamper-proof database, thereby immediately detecting unauthorized alteration. The present protection unit combines role-based access control (RBAC) and attribute-based access control (ABAC) in access control, and executes dynamic authority management according to an attribute of a user, an access location, a time zone, and an urgency level. The present protection unit constantly monitors an access log using an anomaly detection AI, and when detecting an access pattern different from usual (such as download of a large amount of data or access at midnight), immediately blocks a session and transmits an alert to an administrator. Thereby, the present system firmly protects reliability and safety of the medical data by advanced security technology.

The analysis unit can analyze the utterance of the patient using a generative API to accurately grasp a symptom. The analysis unit analyzes the utterance of the patient using the generative API and accurately grasps the symptom. For example, the generative API receives the utterance of the patient as an input and outputs detailed information of the symptom. The generative API converts the utterance of the patient into text data using, for example, speech recognition technology, and analyzes content thereof. In addition, the generative API can extract detailed information of the symptom from the utterance of the patient using natural language processing technology. For example, the generative API analyzes the utterance of the patient and extracts detailed information such as a site, a degree, and an onset time of pain. Thereby, by using the generative API, it is possible to accurately analyze the utterance of the patient and grasp the symptom. Specifically, the present analysis unit is equipped with a specialized model obtained by fine-tuning a pre-trained language model (BERT, GPT, etc.) based on a Transformer architecture with a corpus of a medical domain. The present analysis unit receives an utterance text of the patient as an input token sequence, and calculates a dependency relationship between words in a sentence using a self-attention mechanism. The present analysis unit executes a named entity recognition (NER) task, identifies medical entities such as “abdominal pain (symptom),” “since yesterday (period),” and “severe (degree),” and calculates a reliability score (probability value) for each entity. The present analysis unit executes a relation extraction task for extracting a relationship between the extracted entities, and outputs a causal relationship such as “abdominal pain” occurring “since yesterday” as structured data. When an ambiguous expression (such as “somewhat strange”) is included, the present analysis unit outputs a flag instructing a dialogue management module to generate an additional question, and triggers active information collection for supplementing missing information. Thereby, the present system generates high-precision structured medical information usable for diagnosis support from atypical natural language input.

The creation unit can automatically create the medical record based on the information obtained by the analysis unit using a generative API. The creation unit automatically creates the medical record based on the information obtained by the analysis unit using the generative API. For example, the generative API receives the information obtained from the analysis unit as an input and outputs content of the medical record. The generative API automatically creates the medical record based on the information obtained from the analysis unit using, for example, natural language generation technology. In addition, the generative API can cooperate with an electronic medical record system to automatically update the content of the medical record. For example, the generative API automatically inputs the content of the medical record to the electronic medical record system based on the information obtained from the analysis unit. Thereby, by using the generative API, it is possible to automatically create the medical record. Specifically, the present creation unit receives structured data (key-value pairs of symptom, vital sign, past history, etc.) output by the analysis unit as an input vector. The present creation unit generates a text with a professional and concise style as written by a doctor based on the input vector using a conditional language generation model. The present creation unit automatically classifies and arranges the generated text into each section of a SOAP format (S: Subjective information, O: Objective information, A: Assessment, P: Plan) which is a standard format of a medical record. The present creation unit performs a consistency check using a medical terminology dictionary on a generated draft of the medical record, and automatically corrects a contradiction or a typographical error. The present creation unit transmits generated medical record data as a message in HL7 or FHIR format via an API of the electronic medical record system (EMR), and updates a database. The present creation unit receives a status signal of update completion and records completion of processing in a log. Thereby, the present system significantly reduces a burden of administrative work of the doctor and improves immediacy and accuracy of a medical record.

The conversion unit can convert the explanation of the doctor into the tone of the character using a generative API and convey it to the patient in an easy-to-understand manner. The conversion unit converts the explanation of the doctor into the tone of the character using the generative API and conveys it to the patient in an easy-to-understand manner. For example, the generative API receives the explanation of the doctor as an input and outputs the explanation converted into the tone of the character. The generative API converts the explanation of the doctor into the tone of the character using, for example, natural language generation technology. In addition, the generative API can select an appropriate tone of the character according to the age or the level of understanding of the patient. For example, the generative API selects a friendly tone for children or a polite tone for the elderly. Thereby, by using the generative API, it is possible to convert the explanation of the doctor in an easy-to-understand manner. Specifically, the present conversion unit comprises an encoder configured to receive an utterance text of the doctor as an input and map it to a latent vector space representing semantic content. The present conversion unit holds a style embedding vector defining personality traits and a vocabulary set of a target character (for example, an animal character or an anime character). The present conversion unit combines a latent vector of the utterance of the doctor and the style embedding vector, and inputs them to a decoder, thereby generating a text in which only a writing style is converted while maintaining the semantic content. The present conversion unit performs sentiment analysis on the generated text, and assigns an emotion tag (<happy>, <warning>, etc.) corresponding to content of the text (encouragement, alert, etc.). The present conversion unit inputs this tagged text to a speech synthesis engine, and generates and outputs voice waveform data reproducing voice quality and intonation of the character. The present conversion unit has a switching function of dynamically switching a character model or a vocabulary level (easiness) to be used based on attribute data of the patient. Thereby, the present system lowers a psychological barrier of the patient and improves compliance with medication instruction and lifestyle guidance.

The analysis unit can estimate an emotion of the patient and adjust analysis accuracy of the utterance based on the estimated emotion of the patient. The analysis unit estimates the emotion of the patient and adjusts the analysis accuracy of the utterance based on the estimated emotion of the patient. For example, when the patient feels anxiety, the generative API increases the analysis accuracy of the utterance and extracts more detailed information. In addition, when the patient is relaxed, the generative API can maintain the analysis accuracy of the utterance at a normal level and emphasize natural conversation. Furthermore, when the patient is nervous, the generative API increases the analysis accuracy of the utterance and analyzes carefully to avoid misunderstanding. The estimation of the emotion is realized using an emotion estimation function using, for example, an emotion engine or a generative AI. The generative AI is a text generation AI (for example, LLM), a multimodal generative AI, or the like, but is not limited to such examples. Thereby, by adjusting the analysis accuracy of the utterance based on the emotion of the patient, it is possible to extract more accurate information. Specifically, the present analysis unit comprises a multimodal emotion recognition model configured to receive prosodic features (pitch, intensity, speech rate) of voice data and facial expression features of facial image data as inputs. The present analysis unit outputs an emotional state (anxiety, anger, sadness, joy, etc.) of the patient as a probability distribution from these inputs, and identifies a dominant emotion label and an intensity score (0.0 to 1.0). When the identified emotion score exceeds a predetermined threshold (for example, strong anxiety), the present analysis unit switches to a mode for improving recognition accuracy by expanding a beam search width of a speech recognition engine and searching for more candidates. At the same time, the present analysis unit adjusts an inference parameter (for example, sampling temperature) of a natural language understanding model and changes it to a setting prioritizing elimination of ambiguity. The present analysis unit changes a dialogue strategy according to the emotional state and increases a frequency of confirmation questions, thereby forming a feedback loop ensuring certainty of information. Thereby, the present system corrects disturbance or unclearness of an utterance caused by a psychological state of the patient, and always realizes high-precision information extraction.

The analysis unit can refer to a past medical history of the patient to improve reliability of content of the utterance. The analysis unit refers to the past medical history of the patient and improves the reliability of the content of the utterance. For example, the analysis unit refers to the past medical history of the patient, and increases the reliability of the content of the utterance when there was a similar symptom. In addition, the analysis unit can confirm consistency of the content of the utterance based on the past medical history of the patient and improve the reliability. Furthermore, the analysis unit can refer to the past medical history of the patient, detect a contradiction in the content of the utterance, and improve the reliability. Thereby, by referring to the past medical history, it is possible to improve the reliability of the content of the utterance. Specifically, the present analysis unit adopts a RAG (Retrieval-Augmented Generation) architecture, and searches a vector database using a current utterance of the patient as a query vector. The present analysis unit extracts related documents such as a past medical record, prescription data, and a test result of the patient from the database, and inputs them to a large language model as context information. The present analysis unit calculates semantic similarity and logical consistency between current content of the utterance and a past history, and adds a reliability score of the utterance when the consistency is high. For example, for an utterance “same pain as before,” the present analysis unit links a record of “gastric ulcer” in the past and performs inference to supplement specific symptom details. When there is an obvious contradiction between the content of the utterance and the past history (for example, complaining of pain in an organ that has been removed), the present analysis unit outputs an alert flag suggesting a possibility of phantom limb pain or paramnesia. Thereby, the present system ensures deep insight and high reliability that cannot be obtained by single utterance analysis.

The analysis unit can analyze the utterance of the patient in real time to immediately grasp a change in a symptom. The analysis unit analyzes the utterance of the patient in real time and immediately grasps the change in the symptom. For example, every time the patient speaks, the generative API analyzes in real time and immediately grasps the change in the symptom. In addition, the analysis unit can analyze content of the utterance of the patient in real time and immediately detect a sudden change in the symptom. Furthermore, the analysis unit can analyze the utterance of the patient in real time and immediately grasp a progress status of the symptom. Thereby, by analyzing the utterance in real time, it is possible to immediately grasp the change in the symptom. Specifically, the present analysis unit uses streaming speech recognition technology to divide input voice data into chunks of several milliseconds to several hundred milliseconds and process them sequentially. The present analysis unit continuously inputs text data of a most recent certain time to a natural language processing model using a sliding window method, and monitors a change in a keyword or an expression related to the symptom. The present analysis unit calculates a severity score of the symptom in real time using a time-series data analysis model (RNN or LSTM, etc.), and calculates a time rate of change (derivative value) thereof. When the rate of change exceeds a predetermined threshold (for example, when a complaint of pain increases rapidly), the present analysis unit immediately triggers an emergency event and transmits a push notification to a terminal of a doctor or a nurse. The present analysis unit distributes an analysis result by streaming in real time to a dashboard visualizing transition of conversation, and dynamically updates a trend graph of the symptom. Thereby, the present system supports rapid medical intervention without overlooking a sudden change in a condition or a subtle change in a nuance of the symptom during a medical interview.

The analysis unit can estimate an emotion of the patient and determine a priority of the utterance based on the estimated emotion of the patient. The analysis unit estimates the emotion of the patient and determines the priority of the utterance based on the estimated emotion of the patient. For example, when the patient feels anxiety, the generative API increases the priority of the utterance and preferentially analyzes important information. In addition, when the patient is relaxed, the generative API can maintain the priority of the utterance at a normal level and emphasize natural conversation. Furthermore, when the patient is nervous, the generative API increases the priority of the utterance and analyzes carefully to avoid misunderstanding. The estimation of the emotion is realized using an emotion estimation function using, for example, an emotion engine or a generative AI. The generative AI is a text generation AI (for example, LLM), a multimodal generative AI, or the like, but is not limited to such examples. Thereby, by determining the priority of the utterance based on the emotion of the patient, it is possible to preferentially analyze important information. Specifically, the present analysis unit calculates an emotion score representing “urgency” and “degree of distress” for input utterance data using an emotion analysis model. When a plurality of utterances or information fragments exist, the present analysis unit stores each processing task in a priority queue using the calculated emotion score as a weight. The present analysis unit places an analysis task corresponding to an utterance indicating high anxiety or distress (e.g., “I can't breathe,” “Help me,” etc.) at a head of the queue, and performs scheduling to preferentially allocate calculation resources. In an attention mechanism, the present analysis unit artificially amplifies an attention weight for a token to which a strong emotion is attached, and performs control so that the information is not overlooked in subsequent summary generation or diagnostic inference. When information with high priority is extracted, the present analysis unit bypasses a normal batch processing flow and sends data to the medical record creation unit in an immediate processing flow (Fast Track). Thereby, the present system realizes an adaptive operation of processing important information related to life and safety of the patient with top priority within limited calculation resources.

The analysis unit can improve analysis accuracy of content of the utterance based on a lifestyle habit or environmental information of the patient. The analysis unit improves the analysis accuracy of the content of the utterance based on the lifestyle habit or the environmental information of the patient. For example, the analysis unit considers the lifestyle habit of the patient, understands a background of the content of the utterance, and improves the analysis accuracy. In addition, the analysis unit can refer to the environmental information of the patient and increase reliability of the content of the utterance. Furthermore, the analysis unit can confirm consistency of the content of the utterance based on the lifestyle habit or the environmental information of the patient and improve the analysis accuracy. Thereby, by considering the lifestyle habit or the environmental information, it is possible to improve the analysis accuracy of the content of the utterance. Specifically, the present analysis unit receives lifelog data (activity amount, sleep time, heart rate, room temperature, humidity, etc.) collected from a wearable device or a smart home device of the patient as an auxiliary input. The present analysis unit adopts a multimodal learning model that vectorizes these numerical data and combines them as a context vector with an input embedding layer of a language model. For example, when the patient says “I can't sleep,” the present analysis unit collates it with fact data such as “detection of awakening at midnight” or “high room temperature” in the lifelog data, and performs objective corroboration of the utterance. The present analysis unit refers to environmental information (for example, pollen dispersion amount data), infers that there is a high possibility that an utterance “I have a runny nose” of the patient is allergic, and assigns the inference result as analysis metadata. The present analysis unit performs correlation analysis between lifestyle habit data and the content of the utterance, and drives an inference engine for identifying a risk factor of a lifestyle-related disease. Thereby, the present system realizes multifaceted and high-precision analysis based on objective life data without depending only on a subjective utterance.

The analysis unit can incorporate an opinion of a family member or a caregiver of the patient to improve analysis accuracy of content of the utterance. The analysis unit incorporates the opinion of the family member or the caregiver of the patient and improves the analysis accuracy of the content of the utterance. For example, the analysis unit incorporates the opinion of the family member of the patient and increases reliability of the content of the utterance. In addition, the analysis unit can refer to the opinion of the caregiver of the patient, understand a background of the content of the utterance, and improve the analysis accuracy. Furthermore, the analysis unit can confirm consistency of the content of the utterance based on the opinion of the family member or the caregiver of the patient and improve the analysis accuracy. Thereby, by incorporating the opinion of the family member or the caregiver, it is possible to improve the analysis accuracy of the content of the utterance. Specifically, the present analysis unit applies speaker diarization technology to multi-channel voice data input from a microphone array or the like, and identifies and separates utterances of the patient, the family member, the caregiver, and the doctor. The present analysis unit converts content of an utterance of each speaker into text, tags each utterance source, and stores it in a database. The present analysis unit determines a logical relationship (entailment, contradiction, neutral) between the utterance of the patient (premise) and the utterance of the family member (hypothesis) using a natural language inference (NLI) model. For example, when the utterance of a patient with dementia contradicts a supplementary explanation of the family member, the present analysis unit executes logic to set a high reliability weight for the utterance of the family member and generate integrated symptom information. The present analysis unit uses a multi-document summarization algorithm that combines mutually complementary information while eliminating duplication of information when integrating information from a plurality of information sources. Thereby, the present system accurately reconstructs a true medical condition by integrating information of surrounding supporters even when expressive power of the patient himself/herself is insufficient.

The creation unit can estimate an emotion of the patient and adjust description content of the medical record based on the estimated emotion of the patient. The creation unit estimates the emotion of the patient and adjusts the description content of the medical record based on the estimated emotion of the patient. For example, when the patient feels anxiety, the generative API makes the description content of the medical record detailed to give a sense of security. In addition, when the patient is relaxed, the generative API can maintain the description content of the medical record at a normal level and perform natural description. Furthermore, when the patient is nervous, the generative API makes the description content of the medical record detailed and describes carefully to avoid misunderstanding. The estimation of the emotion is realized using an emotion estimation function using, for example, an emotion engine or a generative AI. The generative AI is a text generation AI (for example, LLM), a multimodal generative AI, or the like, but is not limited to such examples. Thereby, by adjusting the description content of the medical record based on the emotion of the patient, it is possible to give a sense of security. Specifically, the present creation unit inputs an emotion analysis result (emotion label and intensity) received from the analysis unit as a control code of a medical record generation model. When the patient shows high anxiety, the present creation unit gives an instruction to maximize a “Detail” parameter and enable an “Empathy” parameter to the generation model. Thereby, the present creation unit explicitly describes not only a mere list of symptoms but also consideration of the doctor for a complaint of the patient and specific explanation content for relieving anxiety (such as “Please be assured that there is no problem with the test result”) in a “Plan” or “Assessment” column of the medical record. The present creation unit records the emotional state of the patient itself as a “mental finding” in the medical record as structured data, and uses it as reference information at the time of next medical treatment. The present creation unit has an autoregressive verification loop for evaluating whether generated medical record content satisfies an emotional need of the patient, and corrects description as necessary. Thereby, the present system creates a high-quality medical record supporting Patient-Centered Care while maintaining objectivity as a medical record.

The creation unit can refer to a past medical history of the patient when creating the medical record to improve reliability of description content. The creation unit refers to the past medical history of the patient when creating the medical record and improves the reliability of the description content. For example, the creation unit refers to the past medical history of the patient, and increases the reliability of the description content when there was a similar symptom. In addition, the creation unit can confirm consistency of the description content based on the past medical history of the patient and improve the reliability. Furthermore, the creation unit can refer to the past medical history of the patient, detect a contradiction in the description content, and improve the reliability. Thereby, by referring to the past medical history, it is possible to improve the reliability of the description content of the medical record. Specifically, the present creation unit constructs and refers to a knowledge structure in which a past medical history, a medication history, allergy information, and the like of the patient are represented by nodes and edges using knowledge graph technology. When receiving current medical examination data as an input, the present creation unit performs inference on this knowledge graph and automatically identifies relevance to a past disease (recurrence, complication, side effect, etc.). Based on the identified relevance, the present creation unit automatically generates a description based on medical evidence such as “relevance to XX in past history is suspected” in a “Consideration” section of the medical record. The present creation unit has a function of performing a contraindication check between a drug to be prescribed this time and a past side effect history, and automatically inserting a warning sentence in red into the medical record if there is a risk. The present creation unit quantitatively evaluates transition of a time-series symptom (improvement, deterioration, leveling off) by comparison with past data, and embeds a trend analysis result thereof as a graph or a numerical value in the medical record. Thereby, the present system connects fragmentary medical information with a line, and realizes creation of an advanced medical record with continuity and consistency.

The creation unit can reflect a change in a symptom of the patient in real time when creating the medical record. The creation unit reflects the change in the symptom of the patient in real time when creating the medical record. For example, every time the symptom of the patient changes, the generative API updates description content of the medical record in real time. In addition, the creation unit can reflect a sudden change in the symptom of the patient in the medical record in real time. Furthermore, the creation unit can describe a progress status of the symptom of the patient in the medical record in real time. Thereby, by reflecting the change in the symptom in real time, it is possible to describe latest information in the medical record. Specifically, the present creation unit constantly receives a data stream from the analysis unit using a bidirectional communication protocol such as WebSocket or gRPC. Based on the received data, the present creation unit executes an algorithm for performing differential update on a draft version of the medical record held in a memory. For example, when the patient adds a new symptom during a medical interview, the present creation unit immediately adds or corrects a corresponding part and refreshes display on a screen viewed by the doctor. The present creation unit manages a change history of data with a version control system (structure like Git), and records when and what kind of change was made with a timestamp, thereby securing an audit trail. The present creation unit automatically plots real-time numerical data (blood pressure, pulse, etc.) from a vital sign monitor in a predetermined field of the medical record, and performs highlight display at the moment when an abnormal value is detected. Thereby, the present system reflects a situation of a clinical site changing moment by moment in the medical record without delay, and strongly supports decision-making of the doctor.

The creation unit can estimate an emotion of the patient and adjust a description order of the medical record based on the estimated emotion of the patient. The creation unit estimates the emotion of the patient and adjusts the description order of the medical record based on the estimated emotion of the patient. For example, when the patient feels anxiety, the generative API describes important information first to give a sense of security. In addition, when the patient is relaxed, the generative API can describe the medical record in a normal order. Furthermore, when the patient is nervous, the generative API describes important information first and describes carefully to avoid misunderstanding. The estimation of the emotion is realized using an emotion estimation function using, for example, an emotion engine or a generative AI. The generative AI is a text generation AI (for example, LLM), a multimodal generative AI, or the like, but is not limited to such examples. Thereby, by adjusting the description order of the medical record based on the emotion of the patient, it is possible to give a sense of security. Specifically, the present creation unit assigns an importance score and an emotional impact score to each information block (symptom, test result, diagnosis, prescription, etc.) constituting the medical record. The present creation unit uses a Learning to Rank algorithm to determine an optimal information presentation order using an emotional state of the patient (for example, “strong anxiety”) as an input query. In a medical record for a patient with strong anxiety (or a summary for a patient), the present creation unit dynamically generates a “conclusion-first” layout in which a conclusion (such as a benign diagnosis result) is placed at the top and detailed circumstances are placed thereafter. Conversely, when it is estimated that the patient seeks a calm and logical explanation, the present creation unit selects a standard layout according to a chronological order or a medical logical configuration (SOAP order). The present creation unit outputs generated layout information as a style definition in CSS or JSON format, and controls rendering on a display device side. Thereby, the present system reduces a psychological burden on a reader and improves receptivity of information by optimizing the order of information.

The creation unit can consider a lifestyle habit or environmental information of the patient when creating the medical record to enrich description content. The creation unit considers the lifestyle habit or the environmental information of the patient when creating the medical record and enriches the description content. For example, the creation unit considers the lifestyle habit of the patient and enriches the description content of the medical record. In addition, the creation unit can refer to the environmental information of the patient and increase reliability of the description content of the medical record. Furthermore, the creation unit can confirm consistency of the description content of the medical record based on the lifestyle habit or the environmental information of the patient and enrich it. Thereby, by considering the lifestyle habit or the environmental information, it is possible to enrich the description content of the medical record. Specifically, the present creation unit accesses a data lake aggregating big data collected from IoT devices and healthcare apps, and acquires a lifestyle habit profile (smoking, drinking, exercise frequency, sleep quality, etc.) of the patient. The present creation unit inputs these profile data to a natural language generation model, and automatically generates a detailed description including a specific numerical value and a tendency in a “social history” or “lifestyle guidance” section of the medical record. When there is data of “lack of exercise,” for example, the present creation unit inserts individualized guidance content such as “walking once a week is recommended” as a proposal in a “Plan” column. The present creation unit acquires environmental information (epidemic situation of infectious disease in residential area, etc.) from an external API, and automatically adds epidemiological information that can be a basis for diagnosis to a remarks column of the medical record. The present creation unit statistically analyzes a causal relationship between lifestyle habit data and a current symptom, and presents an analysis result thereof (such as “high possibility of headache due to lack of sleep”) as reference information to the doctor. Thereby, the present system realizes comprehensive medical record creation overlooking not only information in a consultation room but also general daily life of the patient.

The creation unit can incorporate an opinion of a family member or a caregiver of the patient when creating the medical record to enrich description content. The creation unit incorporates the opinion of the family member or the caregiver of the patient when creating the medical record and enriches the description content. For example, the creation unit incorporates the opinion of the family member of the patient and enriches the description content of the medical record. In addition, the creation unit can refer to the opinion of the caregiver of the patient and increase reliability of the description content of the medical record. Furthermore, the creation unit can confirm consistency of the description content of the medical record based on the opinion of the family member or the caregiver of the patient and enrich it. Thereby, by incorporating the opinion of the family member or the caregiver, it is possible to enrich the description content of the medical record. Specifically, the present creation unit uses multi-source summarization technology to integrally process an utterance log of the patient himself/herself and provided information (free description of a medical questionnaire or an interview record) from the family member/caregiver. The present creation unit automatically assigns a source tag such as “(heard from wife)” or “(cited from nursing record)” to a description on the medical record in order to clarify a source of information (Source Attribution). When opinions of the patient and the family member differ (for example, a gap in recognition regarding food intake), the present creation unit generates a table or a list describing the difference in a contrast format so that the doctor can objectively grasp a situation. The present creation unit extracts a request from the family member (such as “want to be hospitalized” or “want to care at home”), and highlights it as an important matter in a “Policy” section of the medical record. The present creation unit organizes information from a plurality of concerned parties in chronological order, generates a timeline visualizing a support system surrounding the patient and a change in a home environment, and attaches it to the medical record. Thereby, the present system enables creation of a more three-dimensional and practical medical record incorporating multifaceted perspectives.

The conversion unit can estimate an emotion of the patient and adjust an expression method of the explanation based on the estimated emotion of the patient. The conversion unit estimates the emotion of the patient and adjusts the expression method of the explanation based on the estimated emotion of the patient. For example, when the patient feels anxiety, the generative API provides the explanation in a gentle tone. Also, when the patient is relaxed, the generative API can provide the explanation in a normal tone. Furthermore, when the patient is nervous, the generative API provides the explanation in a calm tone. The estimation of the emotion is realized using an emotion estimation function using, for example, an emotion engine or a generative AI. The generative AI is a text generative AI (e.g., LLM), a multimodal generative AI, or the like, but is not limited to such examples. Thereby, by adjusting the expression method of the explanation based on the emotion of the patient, it is possible to provide a more easy-to-understand explanation. Specifically, the conversion unit dynamically embeds a “Tone and Manner” instruction based on the estimated emotional state into an input prompt to a large language model. When the patient is in an “anxiety” state, the conversion unit imposes a constraint to increase a content rate of positive words and avoid negative expressions or coercive imperative forms for the generated text. In a voice synthesis stage, the conversion unit uses an Emotional TTS (Text-To-Speech) technology to apply voice quality parameters that are soft and inclusive, with a reduced speech speed and suppressed pitch fluctuation, in accordance with an emotion tag of the generated text. Conversely, when the patient shows an attitude of “disregard” or “indifference,” the conversion unit selects a slightly stronger and modulated tone to encourage attention, and converts the explanation into an expression that emphasizes risk information. The conversion unit monitors a reaction of the patient to a conversion result in real time, evaluates whether the emotional state has improved (e.g., reduction of anxiety), and fine-tunes a style of the next utterance. Thereby, the system realizes advanced communication support including not only the content of words but also non-verbal nuances.

The conversion unit can refer to a past medical history of the patient when converting the explanation of the doctor to improve reliability of content of the explanation. The conversion unit refers to the past medical history of the patient when converting the explanation of the doctor to improve the reliability of the content of the explanation. For example, the conversion unit refers to the past medical history of the patient and improves the reliability of the content of the explanation when there was a similar symptom. Also, based on the past medical history of the patient, the conversion unit can confirm consistency of the content of the explanation and improve the reliability. Furthermore, the conversion unit can refer to the past medical history of the patient, detect a contradiction in the content of the explanation, and improve the reliability. Thereby, by referring to the past medical history, it is possible to improve the reliability of the content of the explanation. Specifically, when an explanation text of the doctor is input, the conversion unit searches a past medical history database of the patient and acquires a relevant past experience (e.g., “medicine A prescribed previously”, “surgery three years ago”) as a context. The conversion unit performs a process of automatically replacing or supplementing an abstract explanation of the doctor (“I will prescribe the same medicine as before”) with a specific name (“I will prescribe the medicine called XX that you were taking three years ago”). The conversion unit is equipped with a safety mechanism that temporarily suspends a conversion process and outputs a warning message requesting confirmation to the doctor when the explanation of the doctor contradicts the past history (e.g., recommending a medicine to which the patient has an allergy). The conversion unit learns concepts and terms that the patient had difficulty understanding in the past from the history, and when those terms appear, automatically adds more broken-down metaphorical expressions or references to illustrations. Thereby, the system guarantees conversion into an accurate explanation that leaves no room for misunderstanding, in line with the personal context of the patient.

The conversion unit can reflect a change in a symptom of the patient in real time when converting the explanation of the doctor. The conversion unit reflects the change in the symptom of the patient in real time when converting the explanation of the doctor. For example, the generative API updates the content of the explanation in real time every time the symptom of the patient changes. Also, a sudden change in the symptom of the patient can be reflected in the explanation in real time. Furthermore, a progress status of the symptom of the patient can be reflected in the explanation in real time. Thereby, by reflecting the change in the symptom in real time, it is possible to reflect the latest information in the explanation. Specifically, the conversion unit operates on a low-latency inference engine and monitors real-time input streams from a vital sensor or an image analysis module. When a condition of the patient (e.g., a decrease in oxygen saturation) changes while the doctor is giving an explanation, the conversion unit immediately interrupts a generation process of an explanation text and dynamically inserts additional information or a correction sentence (“Since the value has dropped a little now, I will increase the oxygen”) in line with the current situation. The conversion unit uses a dynamic content generation technology to change text or illustrations displayed on a display for explanation in an animated manner based on real-time symptom data. The conversion unit has a function of detecting a divergence from the latest data when the doctor tries to explain based on old data, and automatically correcting a numerical value to the latest one in the converted utterance for output. Thereby, the system provides a synchronous communication environment without a time lag and improves medical safety.

The conversion unit can estimate an emotion of the patient and adjust an order of the explanation based on the estimated emotion of the patient. The conversion unit estimates the emotion of the patient and adjusts the order of the explanation based on the estimated emotion of the patient. For example, when the patient feels anxiety, the generative API explains important information first to give a sense of security. Also, when the patient is relaxed, the generative API can provide the explanation in a normal order. Furthermore, when the patient is nervous, the generative API explains important information first and explains carefully to avoid misunderstanding. The estimation of the emotion is realized using an emotion estimation function using, for example, an emotion engine or a generative AI. The generative AI is a text generative AI (e.g., LLM), a multimodal generative AI, or the like, but is not limited to such examples. Thereby, by adjusting the order of the explanation based on the emotion of the patient, it is possible to give a sense of security. Specifically, the conversion unit uses a discourse parsing algorithm to divide the content of the explanation of the doctor into semantic units (segments) and identifies a role of each segment (conclusion, reason, detail, exemplification, etc.). The conversion unit reconstructs the segments according to the emotional state of the patient by a discourse planning module. For a patient with strong anxiety, the conversion unit rearranges the segments in the order of “conclusion (it is benign)”->“reason”->“detail” to generate a structure that gives a sense of security at the beginning. Conversely, when the patient is skeptical, the conversion unit adopts an order of “objective data”->“logical reasoning”->“conclusion” to create a structure that enhances persuasiveness. The conversion unit combines texts according to the reconstructed order, corrects conjunctions and demonstratives according to the context, and then performs voice synthesis or text display. Thereby, the system adapts a logical structure of information to a psychological state of the patient and supports effective informed consent.

The conversion unit can consider a lifestyle habit or environmental information of the patient when converting the explanation of the doctor to enrich content of the explanation. The conversion unit considers the lifestyle habit or environmental information of the patient when converting the explanation of the doctor to enrich the content of the explanation. For example, the conversion unit considers the lifestyle habit of the patient and enriches the content of the explanation. Also, the conversion unit can refer to the environmental information of the patient and improve reliability of the content of the explanation. Furthermore, based on the lifestyle habit or environmental information of the patient, the conversion unit can confirm consistency of the content of the explanation and enrich it. Thereby, by considering the lifestyle habit or environmental information, it is possible to enrich the content of the explanation. Specifically, the conversion unit is equipped with a personalization engine that converts a general instruction of the doctor (“Please exercise”) into a specific and actionable action plan (“When you wake up at 6 every morning, let's walk in the nearby park for 15 minutes”) based on lifestyle habit data of the patient (“Wake up at 6 every morning”, “There is a park nearby”). The conversion unit refers to attribute information such as a living environment or an economic situation of the patient, and filters out an unrealizable proposal (e.g., a proposal of “climbing stairs” to a patient living in a house without stairs) or replaces it with an alternative plan. The conversion unit adopts a hybrid method combining rule-based reasoning and generation by a large language model to achieve both medical correctness and suitability for an individual life context. The conversion unit applies a prediction model based on past behavior modification data of the patient to the generated advice, and selects and outputs an expression considered to have the highest execution probability. Thereby, the system provides “striking” advice in line with the actual life of the patient and enhances a therapeutic effect.

The conversion unit can incorporate an opinion of a family member or a caregiver of the patient when converting the explanation of the doctor to enrich content of the explanation. The conversion unit incorporates the opinion of the family member or the caregiver of the patient when converting the explanation of the doctor to enrich the content of the explanation. For example, the conversion unit incorporates the opinion of the family member of the patient and enriches the content of the explanation. Also, the conversion unit can refer to the opinion of the caregiver of the patient and improve reliability of the content of the explanation. Furthermore, based on the opinion of the family member or the caregiver of the patient, the conversion unit can confirm consistency of the content of the explanation and enrich it. Thereby, by incorporating the opinion of the family member or the caregiver, it is possible to enrich the content of the explanation. Specifically, the conversion unit acquires concerns from the family member or the caregiver collected in advance (e.g., “often forgets to take medicine”, “cannot follow dietary restrictions”) from a database and integrates them as supplementary information to the explanation of the doctor. When the doctor explains “Please take the medicine,” the conversion unit converts it into a specific proposal including a viewpoint of the family, such as “To prevent forgetting to take it, which your wife was worried about, let's put it on the table after breakfast,” taking into account information from the family. The conversion unit performs shared optimization of a care plan and automatically generates and adds a message suggesting how the instruction of the doctor should be supported at home (e.g., “Family members, please also pay attention to this point”). The conversion unit also has a function of generating an explanation for the patient and an explanation for the family in different styles, respectively, and distributing them to multiple devices (the patient's tablet and the family's smartphone). Thereby, the system realizes comprehensive care communication involving not only the individual patient but also the entire support network.

The selection unit can estimate an emotion of the patient and select an appropriate expression based on the estimated emotion of the patient. The selection unit estimates the emotion of the patient and selects an appropriate expression based on the estimated emotion of the patient. For example, when the patient feels anxiety, the selection unit selects a gentle expression. Also, when the patient is relaxed, the selection unit can select a normal expression. Furthermore, when the patient is nervous, the selection unit selects a calm expression. The estimation of the emotion is realized using an emotion estimation function using, for example, an emotion engine or a generative AI. The generative AI is a text generative AI (e.g., LLM), a multimodal generative AI, or the like, but is not limited to such examples. Thereby, by selecting an appropriate expression based on the emotion of the patient, it is possible to give a sense of security. Specifically, the selection unit is composed of a generative model that generates a plurality of expression candidates (paraphrases) and a ranking model that selects an optimal solution from those candidates. The selection unit uses an output (emotion vector) from an emotion estimation module as an input feature quantity of the ranking model. When the patient is in an “anxiety” state, the selection unit executes a weighting logic that assigns a high score to a candidate in which the word “pain” is replaced with a softer expression such as “discomfort” or “unpleasantness.” The selection unit incorporates a method of reinforcement learning (RLHF: Reinforcement Learning from Human Feedback) and holds expression patterns that contributed to improvement of the patient's sense of security in past similar cases as a learned policy network. The selection unit continuously verifies effects of different expressions by a method such as an A/B test and autonomously updates an optimal expression dictionary for each emotional state. Thereby, the system automates optimal word choice that always stays close to the patient's feelings.

The selection unit can refer to a past medical history of the patient and select an appropriate expression. The selection unit refers to the past medical history of the patient and selects an appropriate expression. For example, the selection unit refers to the past medical history of the patient and selects an appropriate expression when there was a similar symptom. Also, based on the past medical history of the patient, the selection unit can confirm consistency of expressions and select an appropriate expression. Furthermore, the selection unit can refer to the past medical history of the patient, detect a contradiction in expressions, and select an appropriate expression. Thereby, by referring to the past medical history, it is possible to select an appropriate expression. Specifically, the selection unit applies an algorithm of a recommender system such as collaborative filtering or matrix factorization to predict an “easy-to-understand expression” or a “preferred phrasing” based on a past reaction history of the patient. When there is a history that the patient used the expression “stomach is tingling” in the past, the selection unit preferentially selects a candidate that converts the word “stomachache” of the doctor into “tingling pain” which is the patient's own vocabulary. The selection unit learns a change in a knowledge level of the patient from a long-term medical history, and performs curriculum learning-like control in which a plain expression is selected initially and the expression is gradually shifted to a professional one as treatment progresses. The selection unit has a filtering function of blacklisting an expression that caused misunderstanding in a past explanation and avoiding reuse. Thereby, the system respects a history of dialogue with the patient and contributes to construction of a personalized relationship of trust.

The selection unit can estimate an emotion of the patient and determine a priority of an expression based on the estimated emotion of the patient. The selection unit estimates the emotion of the patient and determines the priority of the expression based on the estimated emotion of the patient. For example, when the patient feels anxiety, the selection unit expresses important information preferentially. Also, when the patient is relaxed, the selection unit can express in a normal order. Furthermore, when the patient is nervous, the selection unit expresses important information preferentially and expresses carefully to avoid misunderstanding. The estimation of the emotion is realized using an emotion estimation function using, for example, an emotion engine or a generative AI. The generative AI is a text generative AI (e.g., LLM), a multimodal generative AI, or the like, but is not limited to such examples. Thereby, by determining the priority of the expression based on the emotion of the patient, it is possible to express important information preferentially. Specifically, the selection unit calculates an importance weight using a saliency map or an attention mechanism for each of information items to be presented (diagnosis name, cause, treatment method, prognosis, etc.). The selection unit takes an emotional state of the patient (e.g., panic state) as an input, and performs dynamic weight adjustment that extremely increases a weight of the most critical information (e.g., “life is not in danger”) and lowers weights of other information, considering a decline in information processing ability. Based on the determined priority, the selection unit simultaneously controls not only a presentation order of information but also multimodal expression parameters such as font size, color, and voice volume. The selection unit performs selection (summarization) of information and outputs a UI control signal such as hiding information with low priority behind a “detail” button. Thereby, the system designs a cognitive flow line to ensure that necessary information is delivered even to an emotionally unstable patient.

The selection unit can consider a lifestyle habit or environmental information of the patient and select an appropriate expression. The selection unit considers the lifestyle habit or environmental information of the patient and selects an appropriate expression. For example, the selection unit considers the lifestyle habit of the patient and selects an appropriate expression. Also, the selection unit can refer to the environmental information of the patient and select an appropriate expression. Furthermore, based on the lifestyle habit or environmental information of the patient, the selection unit can confirm consistency of expressions and select an appropriate expression. Thereby, by considering the lifestyle habit or environmental information, it is possible to select an appropriate expression. Specifically, the selection unit is equipped with a context-aware recommendation engine and selects an optimal message expression according to a current situation (location, time, activity state) of the patient. For example, when it is estimated from GPS information that the patient is at a “workplace,” the selection unit avoids a detailed explanation by voice and selects a concise notification expression by text. The selection unit automatically adjusts a transmission timing of a message or a tone of an expression (“Good morning” or “Good work”) according to the lifestyle habit (night owl, early bird, etc.) of the patient. The selection unit has a logic of inferring a cultural background or regionality (dialect use area, etc.) of the patient from the environmental information and selecting a candidate including a region-specific phrasing or metaphorical expression to create a sense of affinity. Thereby, the system realizes communication without discomfort that naturally blends into a living space of the patient.

The protection unit can estimate an emotion of the patient and adjust a protection level of data based on the estimated emotion of the patient. The protection unit estimates the emotion of the patient and adjusts the protection level of the data based on the estimated emotion of the patient. For example, when the patient feels anxiety, the protection unit increases the protection level of the data to give a sense of security. Also, when the patient is relaxed, the protection unit can maintain a normal protection level. Furthermore, when the patient is nervous, the protection unit increases the protection level of the data and protects carefully to avoid misunderstanding. The estimation of the emotion is realized using an emotion estimation function using, for example, an emotion engine or a generative AI. The generative AI is a text generative AI (e.g., LLM), a multimodal generative AI, or the like, but is not limited to such examples. Thereby, by adjusting the protection level of the data based on the emotion of the patient, it is possible to give a sense of security. Specifically, the protection unit uses a stress value or an anxiety level of the patient calculated by an emotion estimation module as an input parameter of a security policy engine. When the patient shows extreme anxiety or distrust, the protection unit tightens rules of dynamic access control (ABAC), increases a request frequency of multi-factor authentication (MFA) at the time of data viewing, or raises a logging level of an access log to the maximum. The protection unit automatically turns on a privacy filter (peep prevention function) on a screen according to the emotional state of the patient, and controls a granularity of displayed information (detailed display or summary display). The protection unit fosters a psychological sense of security of the patient by visually feeding back that the system is “strictly protected” (highlighting a key icon or notifying “protection mode”). Thereby, the system manages not only technical security strength but also perceived security of the user.

The protection unit can refer to a past medical history of the patient and adjust a protection level of data. The protection unit refers to the past medical history of the patient and adjusts the protection level of the data. For example, the protection unit refers to the past medical history of the patient and adjusts the protection level of the data when there was a similar symptom. Also, based on the past medical history of the patient, the protection unit can confirm consistency of the protection level of the data and adjust it. Furthermore, the protection unit can refer to the past medical history of the patient, detect a contradiction in the protection level of the data, and adjust it. Thereby, by referring to the past medical history, it is possible to adjust the protection level of the data. Specifically, the protection unit evaluates a sensitivity degree of a disease (mental disease, infectious disease, hereditary disease, etc.) included in the past medical history and calculates a risk score. Based on the calculated risk score, the protection unit executes data classification that automatically distributes encryption strength of data or a storage type of a storage destination (on-premise or cloud). When there is a history of being a target of data leakage or unauthorized access in the past, the protection unit deploys an active defense measure such as placing decoy data (honeypot) for the data of the patient. The protection unit learns a past normal access pattern by an anomaly detection model using machine learning, and when detecting an access deviating therefrom (e.g., access from a clinical department not usually referred to), immediately raises the protection level and blocks the access. Thereby, the system realizes detailed and robust data protection according to properties of data and past circumstances.

The protection unit can estimate an emotion of the patient and adjust an access authority of data based on the estimated emotion of the patient. The protection unit estimates the emotion of the patient and adjusts the access authority of the data based on the estimated emotion of the patient. For example, when the patient feels anxiety, the protection unit makes the access authority of the data strict to give a sense of security. Also, when the patient is relaxed, the protection unit can maintain a normal access authority. Furthermore, when the patient is nervous, the protection unit makes the access authority of the data strict and manages carefully to avoid misunderstanding. The estimation of the emotion is realized using an emotion estimation function using, for example, an emotion engine or a generative AI. The generative AI is a text generative AI (e.g., LLM), a multimodal generative AI, or the like, but is not limited to such examples. Thereby, by adjusting the access authority of the data based on the emotion of the patient, it is possible to give a sense of security. Specifically, the protection unit cooperates with an identity management system (IdM) and performs temporary privilege escalation/de-escalation triggered by an emotional state. When the patient is making desperate remarks due to emotional instability, the protection unit temporarily freezes (makes read-only) an authority for data deletion or modification by the patient himself/herself, functioning as a safety device to prevent accidental data loss. When the patient shows a rejection reaction (anger or fear) to a specific medical staff member, the protection unit temporarily restricts an access authority from that staff member to prevent trouble. The protection unit records a history of these authority changes in a tamper-proof ledger such as a blockchain so that legitimacy can be verified later. Thereby, the system prevents data accidents due to emotional trouble and enhances operational stability of the system.

The protection unit can consider a lifestyle habit or environmental information of the patient and adjust a protection level of data. The protection unit considers the lifestyle habit or environmental information of the patient and adjusts the protection level of the data. For example, the protection unit considers the lifestyle habit of the patient and adjusts the protection level of the data. Also, the protection unit can refer to the environmental information of the patient and adjust the protection level of the data. Furthermore, based on the lifestyle habit or environmental information of the patient, the protection unit can confirm consistency of the protection level of the data and adjust it. Thereby, by considering the lifestyle habit or environmental information, it is possible to adjust the protection level of the data. Specifically, the protection unit adopts a geofencing technology using GPS or Wi-Fi connection information, and dynamically switches a security policy based on a physical location of the patient or a terminal. While permitting access with standard authentication when the patient is in a safe area such as “home” or “hospital,” the protection unit requests enforcement of a VPN connection or addition of biometric authentication when the patient is in a high-risk environment such as “cafe” or “overseas.” The protection unit learns a life rhythm (sleeping hours, etc.) of the patient, and determines that access during a time zone when the patient is not usually active is highly likely to be unauthorized access, and maximizes the protection level. The protection unit analyzes ambient environmental sounds (noise level, human voice), and when it is estimated to be a public place, performs control such as switching a screen display to a privacy mode or disabling a voice reading function. Thereby, the system provides an optimal security environment according to the situation without impairing convenience.

The system according to the embodiment is not limited to the above-described examples, and various modifications are possible, for example, as follows. Specifically, each functional unit (analysis unit, creation unit, conversion unit, etc.) of the system is not limited to a form of being aggregated and implemented in a single server, but can adopt a distributed computing architecture in which they are distributed and arranged in a plurality of cloud servers or edge devices (smartphones, tablets, dedicated terminals installed in a hospital). The system can also take a configuration based on a microservice architecture, in which each function is deployed as an independent container (Docker, etc.) and managed by an orchestration tool such as Kubernetes. The system can flexibly select an edge AI configuration in which a part of inference processing of an AI model is executed on a local device of the patient for privacy protection, or a hybrid configuration in which learning processing with a high calculation load is executed on a GPU cluster on a cloud. The system has modularity capable of supporting diverse business models, such as not only being provided as SaaS (Software as a Service) but also package provision to an on-premise environment or function provision as an API.

The analysis unit can consider a background sound or an environmental sound of an utterance when analyzing the utterance of the patient. For example, when the patient is speaking in a noisy environment, the analysis unit removes the background sound using a noise canceling technology and accurately analyzes content of the utterance. Also, when the patient is speaking in a quiet environment, the analysis unit can analyze the content of the utterance without considering the background sound. Furthermore, when the patient is speaking with a specific environmental sound (e.g., a sound in a hospital), the analysis unit can improve reliability of the content of the utterance by reflecting the environmental sound in the analysis. Thereby, the analysis unit can improve analysis accuracy of the content of the utterance by considering the background sound or the environmental sound of the utterance. Specifically, the analysis unit performs Fast Fourier Transform (FFT) on an input signal and executes spectrum analysis in a frequency domain. The analysis unit estimates a stationary noise component and removes it using a spectral subtraction method or a Wiener filter. As more advanced processing, the analysis unit uses a deep learning-based sound source separation model (e.g., CNN having a U-Net structure) to separate “human voice” and “environmental sound (siren, coughing, alarm sound of medical equipment, etc.)” from a mixed sound into separate tracks. The analysis unit inputs the separated environmental sound track to an environmental sound classification model and generates a context tag identifying a current situation (“waiting room”, “inside ambulance”, “home”). By adding this context tag to an input of a language model, the analysis unit increases a prior probability of “high urgency” when, for example, a siren sound of an ambulance is detected, and adjusts an interpretation bias of the content of the utterance. Thereby, the system demonstrates robust recognition performance even under a poor acoustic environment and utilizes the environmental sound itself as a clue for diagnosis.

The selection unit can consider a cultural background or a language difference of the patient and select an appropriate expression. For example, when the patient has a different cultural background, the selection unit selects an expression suitable for the culture. Also, when the patient speaks a different language, the selection unit can select an expression suitable for the language. Furthermore, when the patient speaks multiple languages, the selection unit can select expressions in a plurality of languages. Thereby, by considering the cultural background or the language difference, the selection unit can select an appropriate expression. Specifically, the selection unit integrates a multilingual Neural Machine Translation (NMT) model and a knowledge base storing culture-dependent idioms and manners. The selection unit identifies a “used language” and a “cultural background (nationality, religion, etc.)” from a profile of the patient. In a translation process, the selection unit executes “cultural localization” that selects an appropriate honorific level or euphemistic expression in a target culture, rather than a mere word-for-word translation. For example, for a patient in a cultural sphere where direct reference to death is avoided, the selection unit performs filtering processing of automatically replacing it with a euphemistic expression (“departure”, “pick-up”, etc.). The selection unit uses a cross-lingual embedding space to search for and select an expression vector that is semantically equivalent and has a close emotional nuance even between different languages. Thereby, the system supports smooth medical communication without cultural friction across language barriers.

The protection unit can use a blockchain technology to protect data of the patient. For example, the protection unit records the data of the patient in a blockchain to prevent falsification. Also, the protection unit can record an access history of the data using the blockchain technology and monitor a usage status of the data. Furthermore, the protection unit can safely share the data using the blockchain technology. Thereby, by using the blockchain technology, the protection unit can improve a protection level of the data. Specifically, the protection unit uses a consortium blockchain (Hyperledger Fabric, etc.) or a Layer 2 solution on a public chain to record a hash value of medical data and an access log in a distributed ledger. The protection unit encrypts and stores an actual medical data body (large capacity data) in an off-chain secure storage (IPFS, etc.) and records only the hash value, which is a fingerprint of the data, on-chain, thereby achieving both privacy protection and scalability. The protection unit implements a smart contract to automate and make transparent a process in which the patient himself/herself grants/revokes an access right (token) to his/her own data to/from a third party (another hospital or research institution). The protection unit applies a Zero-Knowledge Proof (ZKP) technology to provide a function of proving only a fact that “a specific condition (e.g., being an adult, having been vaccinated with a specific vaccine) is met” without disclosing a specific medical history of the patient. Thereby, the system restores sovereignty of data to the patient and builds a tamper-proof foundation of trust.

The analysis unit can consider an emotional tone of an utterance when analyzing the utterance of the patient. For example, when the patient is angry, the analysis unit reflects the emotional tone in the analysis. Also, when the patient is sad, the analysis unit can reflect the emotional tone in the analysis. Furthermore, when the patient is happy, the analysis unit can reflect the emotional tone in the analysis. Thereby, by considering the emotional tone of the utterance, the analysis unit can improve analysis accuracy of content of the utterance. Specifically, the analysis unit is equipped with an acoustic analysis engine that extracts prosodic feature quantities such as fundamental frequency (F0), jitter, shimmer, and Harmonics-to-Noise Ratio (HNR) from a voice signal. The analysis unit inputs these feature quantities as time-series data to a Recurrent Neural Network (RNN) and estimates an emotional tone (arousal, valence) for each utterance as a continuous value vector. The analysis unit integrates a result of text analysis (linguistic meaning) and this emotional tone (non-verbal meaning) by a method of late fusion or early fusion. When a tone of sadness or resignation is detected for the word “I'm fine,” for example, the analysis unit executes a logic (irony detection or SOS detection) that denies the linguistic meaning and interprets it as a “state requiring support.” Thereby, the system picks up a true intention hidden behind words and enables understanding at a deeper level.

The creation unit can estimate an emotion of the patient when creating a medical record and adjust description content of the medical record based on the estimated emotion of the patient. For example, when the patient feels anxiety, the generative API details the description content of the medical record to give a sense of security. Also, when the patient is relaxed, the generative API can keep the description content of the medical record at a normal level and perform natural description. Furthermore, when the patient is nervous, the generative API details the description content of the medical record and describes carefully to avoid misunderstanding. Thereby, by adjusting the description content of the medical record based on the emotion of the patient, it is possible to give a sense of security. Specifically, the creation unit adopts a sampling strategy according to an emotion parameter in a decoding process of a generative model (LLM). When the patient is in an anxiety state, the creation unit adjusts parameters (Top-k, Top-p) controlling diversity of generated text to exclude eccentric expressions or ambiguous expressions, and forms a probability distribution in which expressions with high certainty and politeness (e.g., “it is considered that . . . ”, “possibility of . . . is low”) are likely to be selected. The creation unit embeds a result of emotion analysis as metadata in an XML structure of the medical record so that the psychological state of the patient at that time can be reproduced and considered when an AI re-reads this medical record in the future. The creation unit performs similar emotion adjustment in generation of a “medical statement” or an “explanatory document” provided to the patient, and automatically deepens an explanation level of medical terms. Thereby, the system realizes document generation that achieves both accuracy as a record and consideration for the patient.

The conversion unit can estimate an emotion of the patient when converting an explanation of the doctor, and adjust an expression method of the explanation based on the estimated emotion of the patient. For example, when the patient feels anxiety, the generative API gives the explanation in a gentle tone. Also, when the patient is relaxed, the generative API can give the explanation in a normal tone. Furthermore, when the patient is nervous, the generative API gives the explanation in a calm tone. Thereby, by adjusting the expression method of the explanation based on the emotion of the patient, a more easy-to-understand explanation can be given. Specifically, the present conversion unit uses a Prosody Control Model based on deep learning in a text-to-speech (TTS) system. The present conversion unit inputs an estimated emotion label (such as “anxiety” or “relief”) together with input text into a TTS model as a conditioning vector. For an anxious patient, the present conversion unit generates a speech style indicating calmness and acceptance by lowering an intonation at the end of a sentence and taking a longer pause. For a nervous patient, the present conversion unit generates a natural vocalization moderately including a breathing sound (breath) to eliminate mechanical coldness. The present conversion unit performs filtering processing to adjust a spectral envelope of generated speech and impart acoustic characteristics that make the patient auditorily feel “warmth” or “softness”. Thereby, the present system cares for the emotion of the patient not only by content of text but also by resonance of a voice.

The analysis unit can consider a background sound or an environmental sound of the utterance when analyzing the utterance of the patient. For example, when the patient is speaking in a noisy environment, the analysis unit removes the background sound using noise canceling technology to accurately analyze content of the utterance. Also, when the patient is speaking in a quiet environment, the analysis unit can analyze the content of the utterance without considering the background sound. Furthermore, when the patient is speaking with a specific environmental sound (for example, a sound in a hospital), the analysis unit can improve reliability of the content of the utterance by reflecting the environmental sound in the analysis. Thereby, the analysis unit can improve analysis accuracy of the content of the utterance by considering the background sound or the environmental sound of the utterance. Specifically, the present analysis unit uses an Adaptive Digital Filter to update a filter coefficient in real time in accordance with characteristics of environmental noise. For sudden non-stationary noise (door opening/closing sound, dropping sound, etc.), the present analysis unit performs outlier detection in a time domain and masks or interpolates a corresponding section, thereby minimizing an adverse effect on speech recognition. The present analysis unit performs Acoustic Scene Classification to identify a type of the environmental sound (living sound, traffic noise, natural sound) and records a result thereof as metadata. The present analysis unit detects a bioacoustic event such as “coughing sound” or “wheezing (wheezing sound)”, extracts this as objective symptom evidence independent of a linguistic complaint (“painful”), and transmits it to the creation unit. Thereby, the present system picks up information on sounds that do not become words without omission, contributing to improvement in accuracy of diagnosis.

The selection unit can select an appropriate expression in consideration of a cultural background or a language difference of the patient. For example, when the patient has a different cultural background, the selection unit selects an expression suitable for the culture. Also, when the patient speaks a different language, the selection unit can select an expression suitable for the language. Furthermore, when the patient speaks multiple languages, the selection unit can select expressions in a plurality of languages. Thereby, the selection unit can select an appropriate expression by considering the cultural background or the language difference. Specifically, the present selection unit refers to a multilingual Knowledge Graph to map how a certain medical concept is conceptualized in different cultural spheres. The present selection unit identifies a difference between a culture preferring a numerical scale (1-10) and a culture preferring a visual analog scale (facial expression) in, for example, an expression of “pain”, and automatically switches a presentation format on a UI. The present selection unit is equipped with a language model supporting code switching (a phenomenon of speaking by mixing a plurality of languages during conversation), and generates a response expression in an appropriate language without interrupting a context even when a bilingual patient speaks while switching languages. The present selection unit holds information regarding a religious taboo (medicine or treatment derived from a specific animal) as a knowledge base, and performs filtering of expressions and presentation of alternatives so that a proposed content does not conflict with a cultural/religious belief of the patient. Thereby, the present system provides an interface optimized for each patient having a diverse background in a globalizing medical field.

The protection unit can use blockchain technology to protect data of the patient. For example, the protection unit records the data of the patient in a blockchain to prevent falsification. Also, the protection unit can record an access history of the data using the blockchain technology to monitor a usage status of the data. Furthermore, the protection unit can safely perform sharing of the data using the blockchain technology. Thereby, the protection unit can improve a protection level of the data by using the blockchain technology. Specifically, the present protection unit combines Decentralized Identity (DID) technology and the blockchain to realize a Self-Sovereign Identity (SSI) model in which the patient manages a “key” of his/her own medical data. The present protection unit controls automatic and secure data distribution based on a predefined condition (“share only prescription data”, “valid for 3 days”, etc.) using a smart contract in data sharing among different stakeholders such as a medical institution, an insurance company, and a pharmacy. The present protection unit irreversibly links all past change histories by a hash chain structure of data, making it mathematically impossible for even a malicious administrator to secretly falsify the data. The present protection unit provides a node for an auditing organization to ensure transparency enabling a compliance audit in real time. Thereby, the present system overcomes vulnerability of a conventional centralized management type database and establishes reliability as a next-generation medical data platform.

The analysis unit can consider an emotional tone of the utterance when analyzing the utterance of the patient. For example, when the patient is angry, the analysis unit reflects the emotional tone in the analysis. Also, when the patient is sad, the analysis unit can reflect the emotional tone in the analysis. Furthermore, when the patient is happy, the analysis unit can reflect the emotional tone in the analysis. Thereby, the analysis unit can improve analysis accuracy of content of the utterance by considering the emotional tone of the utterance. Specifically, the present analysis unit uses a dual encoder model in which a Convolutional Neural Network (CNN) taking a spectrogram image of speech as an input and a Transformer taking a text embedding as an input are arranged in parallel. The present analysis unit integrates outputs thereof in a Concatenation Layer to perform emotion class classification and perform regression analysis on “urgency” or “seriousness” of the utterance. The present analysis unit performs advanced context understanding to detect a divergence between a speech feature (component of laughter) and a text feature (meaning of pain) when, for example, the patient says “it hurts” while laughing (bitter smile), and interpret this as “mild pain” or “hiding embarrassment”. The present analysis unit also has a function of tracking a change in the emotional tone in time series (change from anger to gratitude, etc.) and quantifying and feeding back how a response of the doctor affected the emotion of the patient. Thereby, the present system reproduces an ability to read a delicate atmosphere like a human by AI, and dramatically improves quality of the analysis.

A flow of processing of Example of the Embodiment will be briefly described below. Specifically, a data processing pipeline in the present system is configured as a series of sequential and parallel processes in which a plurality of dedicated modules operate in cooperation from acquisition of input data to final output generation. The present process is designed to achieve both responsiveness in a medical field and accuracy of a record by appropriately combining stream processing emphasizing real-time performance and batch processing emphasizing accuracy. Each step shown below is controlled by a software program executed on a hardware resource including a central processing unit (CPU) and a graphics processing unit (GPU).

Step 1: The analysis unit analyzes the utterance of the patient. The utterance of the patient includes voice data and text data. The analysis unit converts the utterance of the patient into text data using speech recognition technology and analyzes content thereof. Also, the analysis unit can analyze the utterance of the patient using the generative API to accurately grasp a symptom. Step 2: The creation unit automatically creates a medical record based on information obtained by the analysis unit. The medical record describes the symptom of the patient, content of the utterance, a medical history, and the like. The creation unit can also automatically create the medical record based on the information obtained by the analysis unit using the generative API. Step 3: The conversion unit converts the explanation of the doctor into the tone of the character based on the medical record created by the creation unit. The conversion unit can convert the explanation of the doctor into the tone of the character using the generative API to convey the explanation to the patient in an easy-to-understand manner. Specifically, in Step 1, the analysis unit performs A/D conversion on an analog audio signal acquired from an input device such as a microphone and buffers it as digital waveform data. The analysis unit performs preprocessing (noise removal, etc.) on this data, then inputs it to a speech recognition engine and a natural language understanding model, and generates structured data (JSON object, etc.) including an intention of the utterance, an emotion, and a medical entity (symptom, part, etc.) to expand it on a memory. In Step 2, the creation unit takes the structured data generated in Step 1 as an input, and generates a medical record draft complying with a SOAP format using a medical knowledge base and a template engine, or a large-scale language model. The creation unit performs a logic check and terminology unification processing on the generated draft, and commits determined medical record data to a database of an electronic medical record system as a transaction. In Step 3, the conversion unit takes an explanation text input (or speech-recognized) by the doctor and a level of understanding/emotion parameter of the patient obtained in Step 1 as inputs. The conversion unit simplifies and characterizes the text using a style transfer model, and further generates a speech waveform using a speech synthesis engine. The conversion unit outputs synthesized speech and visual auxiliary information (avatar animation, etc.) to a display and a speaker of a patient terminal as a final output, and completes a series of processing.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of a plurality of elements including the above-described analysis unit, creation unit, conversion unit, selection unit, and protection unit is implemented in, for example, at least one of the smart device 14 and the data processing apparatus 12. For example, the analysis unit is implemented by a processor 46 of the smart device 14 and analyzes an utterance of a patient. The creation unit is implemented by a specific processing unit 290 of the data processing apparatus 12 and automatically creates a medical record. The conversion unit is implemented by a control unit 46A of the smart device 14 and converts an explanation of a doctor into a tone of a character. The selection unit is implemented by the specific processing unit 290 of the data processing apparatus 12 and selects an expression corresponding to an age or a level of understanding of the patient. The protection unit is implemented by the specific processing unit 290 of the data processing apparatus 12 and ensures accuracy of medical data and protects privacy. The correspondence relationship between each unit and the device or the control unit is not limited to the above-described example, and various modifications are possible.

### Second Embodiment

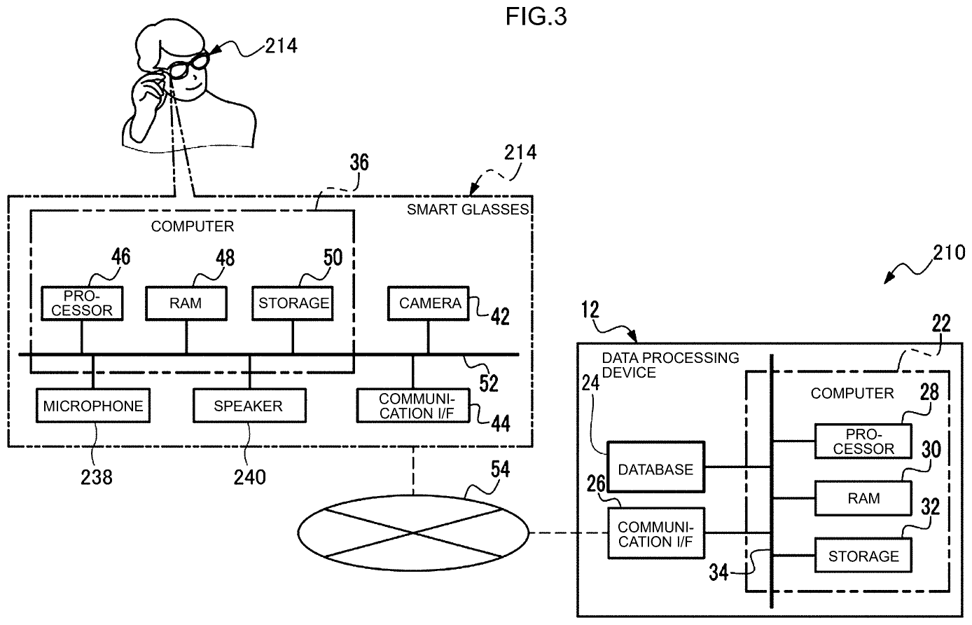

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

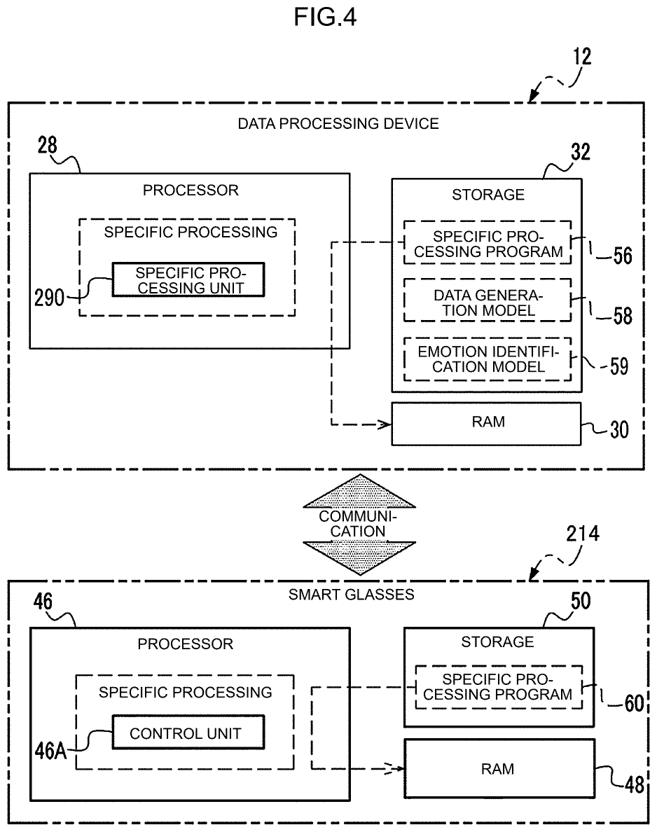

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of a plurality of elements including the above-described analysis unit, creation unit, conversion unit, selection unit, and protection unit is implemented in, for example, at least one of the smart glasses 214 and the data processing apparatus 12. For example, the analysis unit is implemented by a processor 46 of the smart glasses 214 and analyzes an utterance of a patient. The creation unit is implemented by a specific processing unit 290 of the data processing apparatus 12 and automatically creates a medical record. The conversion unit is implemented by a control unit 46A of the smart glasses 214 and converts an explanation of a doctor into a tone of a character. The selection unit is implemented by the specific processing unit 290 of the data processing apparatus 12 and selects an expression corresponding to an age or a level of understanding of the patient. The protection unit is implemented by the specific processing unit 290 of the data processing apparatus 12 and ensures accuracy of medical data and protects privacy. The correspondence relationship between each unit and the device or the control unit is not limited to the above-described example, and various modifications are possible.

### Third Embodiment

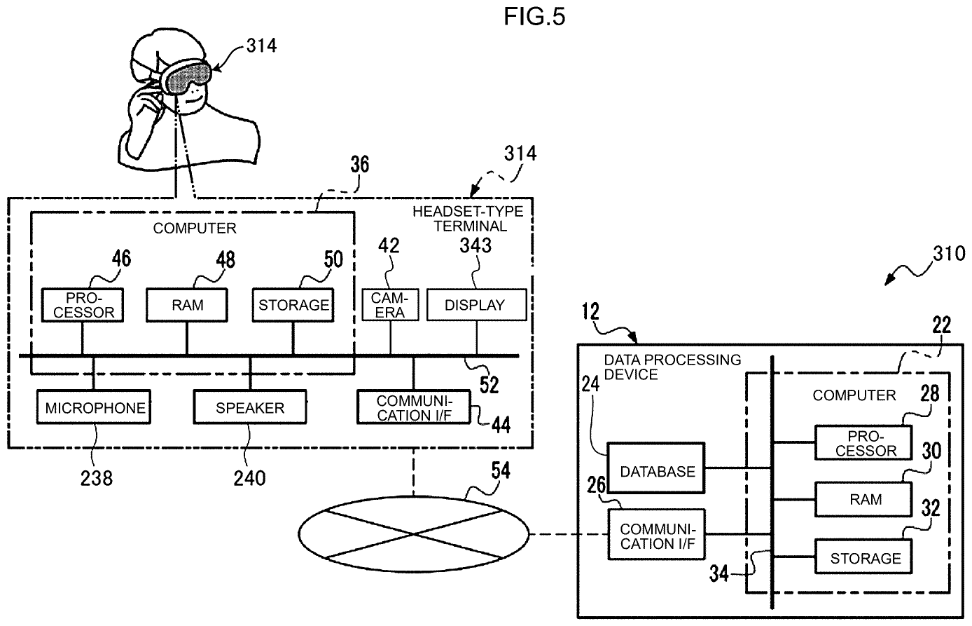

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

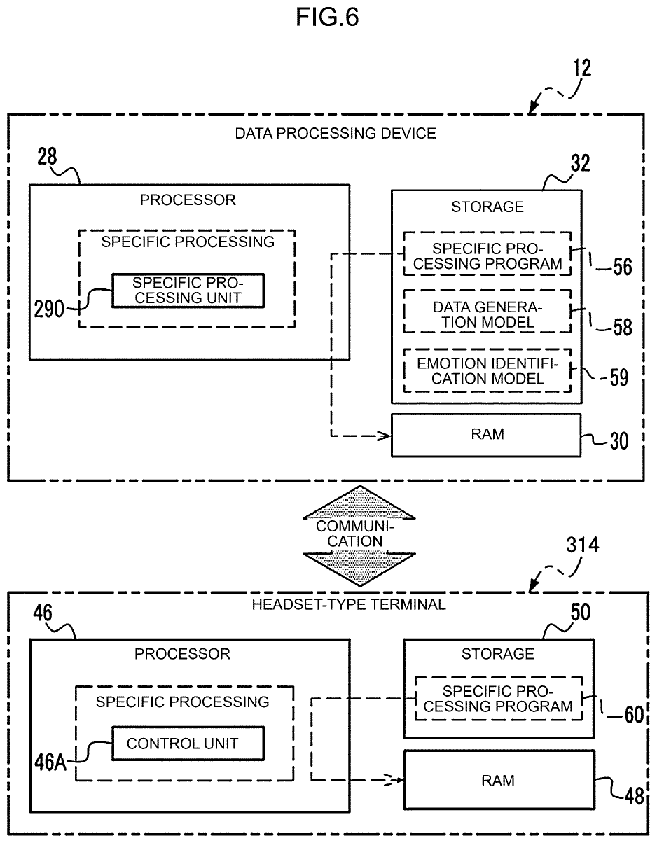

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of a plurality of elements including the above-described analysis unit, creation unit, conversion unit, selection unit, and protection unit is implemented in, for example, at least one of the headset-type terminal 314 and the data processing apparatus 12. For example, the analysis unit is implemented by a processor 46 of the headset-type terminal 314 and analyzes an utterance of a patient. The creation unit is implemented by a specific processing unit 290 of the data processing apparatus 12 and automatically creates a medical record. The conversion unit is implemented by a control unit 46A of the headset-type terminal 314 and converts an explanation of a doctor into a tone of a character. The selection unit is implemented by the specific processing unit 290 of the data processing apparatus 12 and selects an expression corresponding to an age or a level of understanding of the patient. The protection unit is implemented by the specific processing unit 290 of the data processing apparatus 12 and ensures accuracy of medical data and protects privacy. The correspondence relationship between each unit and the device or the control unit is not limited to the above-described example, and various modifications are possible.

### Fourth Embodiment

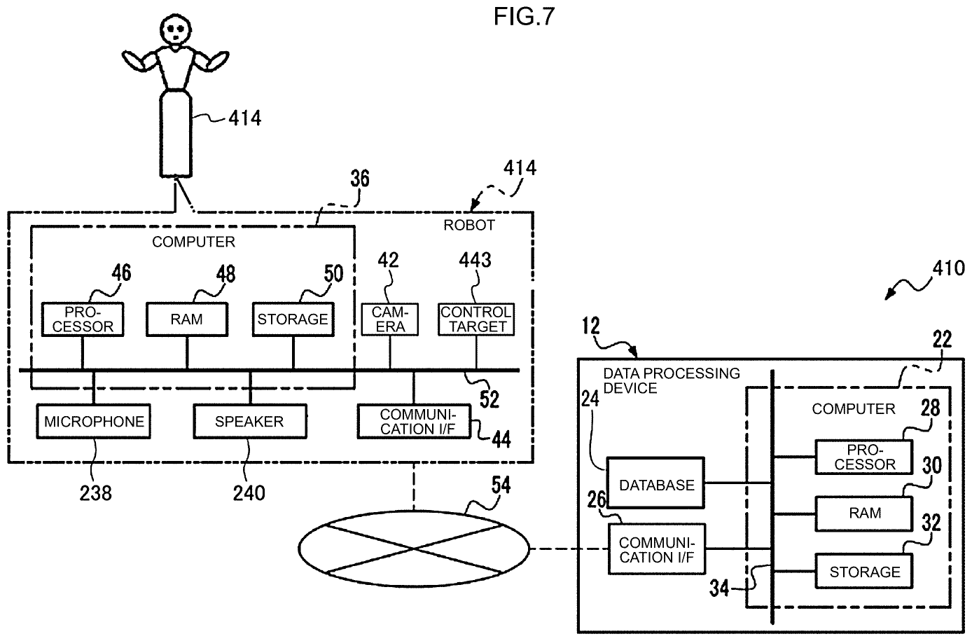

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

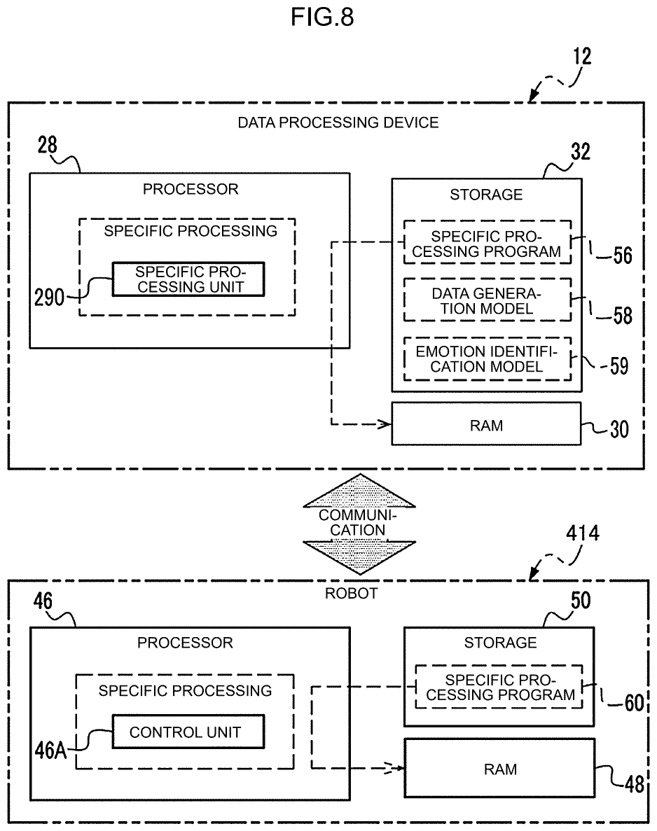

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of a plurality of elements including the above-described analysis unit, creation unit, conversion unit, selection unit, and protection unit is implemented in, for example, at least one of the robot 414 and the data processing apparatus 12. For example, the analysis unit is implemented by a processor 46 of the robot 414 and analyzes an utterance of a patient. The creation unit is implemented by a specific processing unit 290 of the data processing apparatus 12 and automatically creates a medical record. The conversion unit is implemented by a control unit 46A of the robot 414 and converts an explanation of a doctor into a tone of a character. The selection unit is implemented by the specific processing unit 290 of the data processing apparatus 12 and selects an expression corresponding to an age or a level of understanding of the patient. The protection unit is implemented by the specific processing unit 290 of the data processing apparatus 12 and ensures accuracy of medical data and protects privacy. The correspondence relationship between each unit and the device or the control unit is not limited to the above-described example, and various modifications are possible.

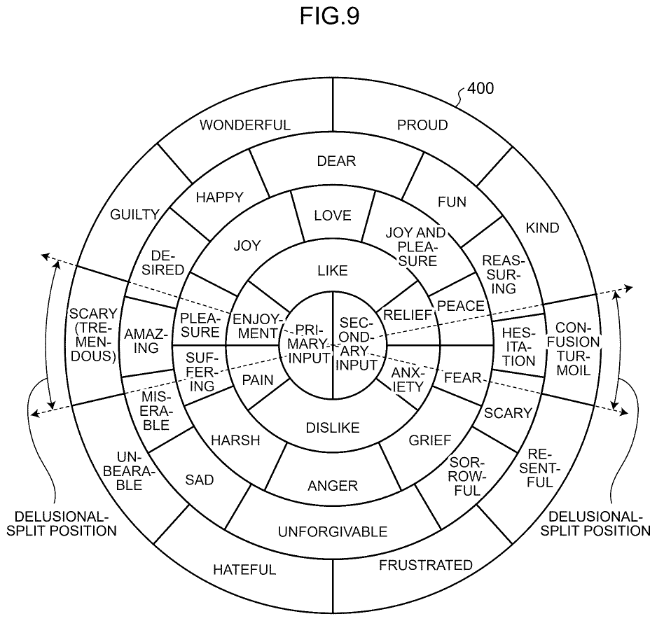

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

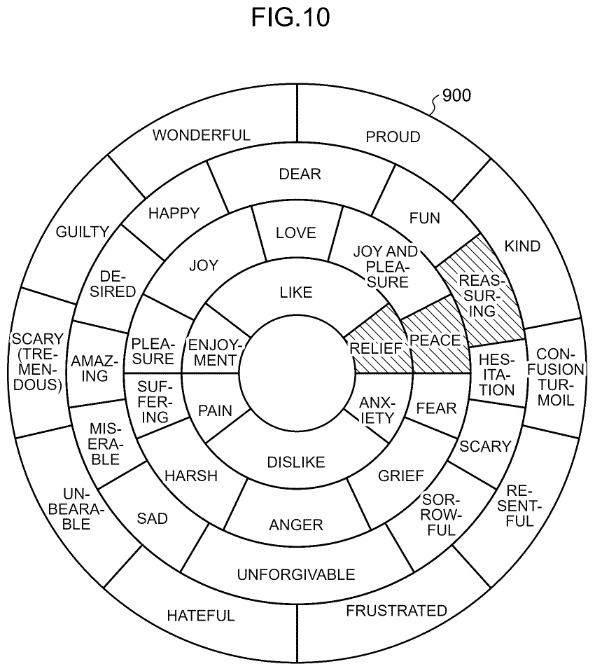

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: an analysis unit configured to analyze an utterance of a patient; a creation unit configured to automatically create a medical record based on information obtained by the analysis unit; and a conversion unit configured to convert an explanation of a doctor into a tone of a character based on the medical record created by the creation unit.
(Supplementary Note 2) The system according to Supplementary Note 1, further comprising a selection unit configured to select an expression corresponding to an age or a level of understanding of the patient.
(Supplementary Note 3) The system according to Supplementary Note 1, further comprising a protection unit configured to ensure accuracy of medical data and protect privacy.
(Supplementary Note 4) The system according to Supplementary Note 1, wherein the analysis unit is configured to analyze the utterance of the patient using a generative API to accurately grasp a symptom.
(Supplementary Note 5) The system according to Supplementary Note 1, wherein the creation unit is configured to automatically create the medical record based on the information obtained by the analysis unit using a generative API.
(Supplementary Note 6) The system according to Supplementary Note 1, wherein the conversion unit is configured to convert the explanation of the doctor into the tone of the character using a generative API to convey the explanation to the patient in an easy-to-understand manner.
(Supplementary Note 7) The system according to Supplementary Note 1, wherein the analysis unit is configured to estimate an emotion of the patient and adjust analysis accuracy of the utterance based on the estimated emotion of the patient.
(Supplementary Note 8) The system according to Supplementary Note 1, wherein the analysis unit is configured to refer to a past medical history of the patient to improve reliability of content of the utterance.
(Supplementary Note 9) The system according to Supplementary Note 1, wherein the analysis unit is configured to analyze the utterance of the patient in real time to immediately grasp a change in a symptom.
(Supplementary Note 10) The system according to Supplementary Note 1, wherein the analysis unit is configured to estimate an emotion of the patient and determine a priority of the utterance based on the estimated emotion of the patient.
(Supplementary Note 11) The system according to Supplementary Note 1, wherein the analysis unit is configured to improve analysis accuracy of content of the utterance based on a lifestyle habit or environmental information of the patient.
(Supplementary Note 12) The system according to Supplementary Note 1, wherein the analysis unit is configured to incorporate an opinion of a family member or a caregiver of the patient to improve analysis accuracy of content of the utterance.
(Supplementary Note 13) The system according to Supplementary Note 1, wherein the creation unit is configured to estimate an emotion of the patient and adjust description content of the medical record based on the estimated emotion of the patient.
(Supplementary Note 14) The system according to Supplementary Note 1, wherein the creation unit is configured to refer to a past medical history of the patient when creating the medical record to improve reliability of description content.
(Supplementary Note 15) The system according to Supplementary Note 1, wherein the creation unit is configured to reflect a change in a symptom of the patient in real time when creating the medical record.
(Supplementary Note 16) The system according to Supplementary Note 1, wherein the creation unit is configured to estimate an emotion of the patient and adjust a description order of the medical record based on the estimated emotion of the patient.
(Supplementary Note 17) The system according to Supplementary Note 1, wherein the creation unit is configured to consider a lifestyle habit or environmental information of the patient when creating the medical record to enrich description content.
(Supplementary Note 18) The system according to Supplementary Note 1, wherein the creation unit is configured to incorporate an opinion of a family member or a caregiver of the patient when creating the medical record to enrich description content.
(Supplementary Note 19) The system according to Supplementary Note 1, wherein the conversion unit is configured to estimate an emotion of the patient and adjust an expression method of the explanation based on the estimated emotion of the patient.
(Supplementary Note 20) The system according to Supplementary Note 1, wherein the conversion unit is configured to refer to a past medical history of the patient when converting the explanation of the doctor to improve reliability of content of the explanation.
(Supplementary Note 21) The system according to Supplementary Note 1, wherein the conversion unit is configured to reflect a change in a symptom of the patient in real time when converting the explanation of the doctor.
(Supplementary Note 22) The system according to Supplementary Note 1, wherein the conversion unit is configured to estimate an emotion of the patient and adjust an order of the explanation based on the estimated emotion of the patient.
(Supplementary Note 23) The system according to Supplementary Note 1, wherein the conversion unit is configured to consider a lifestyle habit or environmental information of the patient when converting the explanation of the doctor to enrich content of the explanation.
(Supplementary Note 24) The system according to Supplementary Note 1, wherein the conversion unit is configured to incorporate an opinion of a family member or a caregiver of the patient when converting the explanation of the doctor to enrich content of the explanation.
(Supplementary Note 25) The system according to Supplementary Note 2, wherein the selection unit is configured to estimate an emotion of the patient and select an appropriate expression based on the estimated emotion of the patient.
(Supplementary Note 26) The system according to Supplementary Note 2, wherein the selection unit is configured to refer to a past medical history of the patient and select an appropriate expression.
(Supplementary Note 27) The system according to Supplementary Note 2, wherein the selection unit is configured to estimate an emotion of the patient and determine a priority of the expression based on the estimated emotion of the patient.
(Supplementary Note 28) The system according to Supplementary Note 2, wherein the selection unit is configured to consider a lifestyle habit or environmental information of the patient and select an appropriate expression.
(Supplementary Note 29) The system according to Supplementary Note 3, wherein the protection unit is configured to estimate an emotion of the patient and adjust a protection level of data based on the estimated emotion of the patient.
(Supplementary Note 30) The system according to Supplementary Note 3, wherein the protection unit is configured to refer to a past medical history of the patient and adjust a protection level of data.
(Supplementary Note 31) The system according to Supplementary Note 3, wherein the protection unit is configured to estimate an emotion of the patient and adjust an access authority of data based on the estimated emotion of the patient.
(Supplementary Note 32) The system according to Supplementary Note 3, wherein the protection unit is configured to consider a lifestyle habit or environmental information of the patient and adjust a protection level of data.

What is claimed is:

1. A system comprising:
circuitry configured to:
receive, via a communication interface coupled to a packet-switched network, input data comprising voice data captured by a client terminal;
convert the voice data to text data using an automatic speech recognition model;
apply a natural language understanding model based on a Transformer architecture to the text data to extract structured entity data comprising a plurality of entity-attribute pairs;
generate, using a language generation model, a structured record in a predetermined format based on the structured entity data;
receive, via the communication interface, explanation data comprising a natural-language text input;
apply a style transfer model to the explanation data to generate converted output data, the style transfer model converting a writing style of the explanation data based on a persona vector while maintaining semantic content of the explanation data; and
transmit the converted output data to the client terminal via the communication interface for output to a user.

2. The system according to claim 1, wherein the input data comprises an utterance of a patient describing a symptom, and wherein the structured entity data comprises at least one of a symptom name, a body site, a severity degree, or an onset time extracted from the utterance.

3. The system according to claim 1, wherein the structured record comprises a medical record in a SOAP format including subjective data, objective data, assessment, and a plan.

4. The system according to claim 1, wherein the explanation data comprises an explanation of a doctor, and wherein the persona vector defines vocabulary, sentence ending style, and tone of a specified character.

5. The system according to claim 1, wherein the circuitry is further configured to estimate a comprehension level of the user based on attribute information of the user, and to dynamically add a constraint condition corresponding to the estimated comprehension level to an input prompt of the language generation model to adjust a vocabulary complexity of the converted output data.

6. The system according to claim 1, wherein the circuitry is further configured to apply a personal information protection filter to the structured entity data to detect personally identifiable information, and to perform at least one of masking or tokenization processing on the detected personally identifiable information before generating the structured record.

7. The system according to claim 1, wherein the circuitry is further configured to execute a named entity recognition task on the text data to identify entity labels and calculate a reliability score for each identified entity, and to output a flag instructing generation of an additional query when an ambiguous expression is detected in the text data.

8. The system according to claim 1, wherein the circuitry is further configured to generate the structured record by inputting the structured entity data as an input vector into a conditional language generation model, and to perform a consistency check on the generated structured record using a terminology dictionary.

9. The system according to claim 1, wherein the style transfer model comprises an encoder configured to map the explanation data to a latent vector space representing semantic content, and a decoder configured to combine a latent vector of the explanation data with the persona vector to generate the converted output data in which only a writing style is converted while maintaining the semantic content.

10. The system according to claim 1, wherein the circuitry is further configured to estimate an emotion of the user by applying an emotion identification model to at least one of prosodic features of the voice data or facial expression features of image data received from the client terminal, and to adjust an inference parameter of the natural language understanding model based on the estimated emotion.

11. The system according to claim 1, wherein the circuitry is further configured to search a vector database using a retrieval-augmented generation architecture with the text data as a query vector to retrieve related past records, and to input the retrieved past records as context information into the natural language understanding model to calculate a reliability score of the text data based on consistency with the past records.

12. The system according to claim 1, wherein the circuitry is further configured to process the voice data in real time using streaming speech recognition to divide the voice data into sequential chunks, and to monitor changes in keyword frequency within a sliding window of the text data using a time-series analysis model to detect a rate of change exceeding a predetermined threshold.

13. The system according to claim 1, wherein the circuitry is further configured to calculate an emotion score representing urgency from the input data using an emotion analysis model, and to store analysis tasks in a priority queue weighted by the calculated emotion score such that input data associated with a high urgency score is processed with higher priority.

14. The system according to claim 1, wherein the circuitry is further configured to receive auxiliary input data comprising at least one of activity amount, sleep time, or environmental sensor data from an IoT device associated with the user, and to combine the auxiliary input data as a context vector with an input embedding layer of the natural language understanding model to corroborate the text data with objective data.

15. The system according to claim 1, wherein the circuitry is further configured to apply speaker diarization to multi-channel voice data to identify and separate utterances from a plurality of speakers, and to determine a logical relationship between utterances of different speakers using a natural language inference model to generate integrated entity data.

16. The system according to claim 1, wherein the circuitry is further configured to estimate an emotion of the user and to dynamically embed an instruction based on the estimated emotion into an input prompt to the style transfer model, such that when the estimated emotion indicates anxiety, the style transfer model generates the converted output data with an increased content rate of positive words and avoidance of negative expressions.

17. The system according to claim 1, wherein the circuitry is further configured to perform spectral analysis on the voice data to estimate a stationary noise component, remove the estimated noise component using a spectral subtraction method, and apply a deep learning-based sound source separation model to separate a human voice track from an environmental sound track in the voice data.

18. A system comprising:
a communication interface coupled to a packet-switched network and configured to communicate with a client terminal, the client terminal comprising a microphone configured to capture voice data and at least one of a display or a speaker configured to output data to a user;
a processor comprising at least one of a CPU, a GPU, or a TPU;
a RAM connected to the processor;
a memory storing a data generation model obtained by performing deep learning on a neural network and an emotion identification model;
a database; and
circuitry configured to:
receive, via the communication interface, the voice data captured by the microphone of the client terminal;
convert the voice data to text data using an automatic speech recognition model executed on the RAM by the processor;
apply a natural language understanding model based on a Transformer architecture to the text data to extract structured entity data comprising a plurality of entity-attribute pairs;
store the structured entity data in the database;
generate, using the data generation model, a structured record in a predetermined format based on the structured entity data retrieved from the database;
store the structured record in the database;
estimate an emotion of the user by applying the emotion identification model to at least one of prosodic features of the voice data or facial expression features of image data received from the client terminal;
receive, via the communication interface, explanation data comprising a natural-language text input;
apply a style transfer model to the explanation data to generate converted output data, the style transfer model converting a writing style of the explanation data based on a persona vector and the estimated emotion while maintaining semantic content of the explanation data; and
transmit the converted output data to the client terminal via the communication interface, the converted output data causing the client terminal to output the converted output data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a system, the method comprising:
receiving, via a communication interface coupled to a packet-switched network, input data comprising voice data captured by a client terminal;
converting the voice data to text data using an automatic speech recognition model;
applying a natural language understanding model based on a Transformer architecture to the text data to extract structured entity data comprising a plurality of entity-attribute pairs;
generating, using a language generation model, a structured record in a predetermined format based on the structured entity data;
receiving, via the communication interface, explanation data comprising a natural-language text input;
applying a style transfer model to the explanation data to generate converted output data, the style transfer model converting a writing style of the explanation data based on a persona vector while maintaining semantic content of the explanation data; and
transmitting the converted output data to the client terminal via the communication interface for output to a user.
